SEQUENCE LISTING (15 sequences)

SEQ ID NO: 1 - DNA, 1486 bp, Cucumis sativus
aagctttacaaattagggttactttattcattttcatccattctctttattgttaaattttgtacatttattcaataatattatatgtttattacaaattctcactttcttattcatacctattcactcaagcctttaccatcttccttttctatttcaatactatttctacttcatttttcacgtttttaacatctttctttatttcttgtccacttcgtttagggatgcctaatgtcccaaatttcatctctcgtagtaacacaaaaccaatgtaatgctacttctctctacatttttaatacaaataaagtgaaacaaaatatctataaataaacaaatatatatattttgttagacgctgtctcaacccatcaattaaaaaattttgttatatttctactttacctactaaatttgtttctcatatttaccttttaacccccacaaaaaaaaattataaaaaagaaagaaaaaagctaaaccctatttaaatagctaactataagatcttaaaattatcctcatcagtgtatagtttaattggttattaacttataacattatatatctatgacatatactctctcctagctatttctcacattttttaacttaagaaaatagtcataacatagtctaaaattcaaacatccacatgctctaatttgattaacaaaaagttagaaatatttatttaaataaaaaagactaataaatatataaaatgaatgttcatacgcagacccatttagagatgagtatgctttcacatgctgagattattttcaaaactaaggttgtagcaatattaaatcaataaaattattataaataacaaaattaacctgctcgtgtttgctgtatatgggaggctacaaaataaattaaactaaagatgattatgttttagacattttttctatctgtattagtttatacatattaattcaggagctgcacaacccaattctattttcgttccttggtggctgggtttctcacaaggttcaatagtcaatattaggttttattggacttttaatagtatcaaacaaatctatgtgtgaacttaaaaattgtattaaatatttagggtaacctgttgccgtttttagaataatgtttcttcttaatacacgaaagcgtattgtgtattcattcatttggcgcctcacatgcttcggttggctcgctttagtctctgccttctttgtatattgtactccccctcttcctatgccacgtgttctgagcttaacaagccacgttgcgtgccattgccaaacaagtcattttaacttcacaaggtccgatttgacctccaaaacaacgacaagtttccgaacagtcgcgaagatcaagggtataatcgtctttttgaattctatttctctttatttaatagtccctctcgtgtgatagtttttaaaagatttttaaaacgtagctgctgtttaagtaaatcccagtccttcagtttgta

SEQ ID NO: 2 - DNA, 747 bp, Arabidopsis thaliana
atggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcccccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattag

SEQ ID NO: 3 - DNA, 316 bp, Cucumis sativus
cgcctcacatgcttcggttggctcgctttagtctctgccttctttgtattttgtactccccctcttcctgtgccacgtgttctgagcttaacaagccacgttgcgtgccattgccaaacgagtcattttaacttcacaaggtccgatttgacctccaaaacaacgacaagtttccgaacagtcgcgaagatcaagggtataatcgtctttttgaattctatttctctttatttaatagtccctctcgtgtgatagttttttaaagatttttaaaacgtagctgctgtttaagtaaatcccagtccttcagtttgta

SEQ ID NO: 4 - DNA, 913 bp, Arabidopsis thaliana
aagcttgctgcagctttgacgacaagtaggtttgttcaattagtagcatctttacaatgtaaagcttttctcttcatttctctttctttctttctttcatgcaagttctattgattagaaaccaaaaccatgacatatcacattttcaatttttaaccccaagtttcccaaaaaggtaatcacaattactttccttggtcatttcttctggttcaagcatgacatgaacaggcaataaataagttgagattttgatcacagtaactgatacttgaatcgaatcatttagattttttttttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctatactattcaagaaactcgaagaacattctctcttcatcctctaacttccctataaatatgtcctttgctaatcagatcaaatcagcaggaaaatcaagaaccaaaagtctcccgaaaagcaacgaca

SEQ ID NO: 5 - DNA, 1977 bp, Arabidopsis thaliana
aactgcgaaaaaagggagcaataaagttcagaaacaagcccttgactgtatgttgagaagaaataaacattacctgccatggtctttcccagtcaagtggcataggccaagctccaaaccatcctccaataattgctccatatgctggaactaagagcatatgttctataattccgattggtctaaaagaaaaaaaaaagatggattaacaacaacataaagtgtgtgaattatcatactaggtagcataagtgacttactttagtgaagcaaagatacgatgccaatctatccatgatgcacctaaaactgcagttgctggtacaacctatgaaaatcaaaatcatcagacaccatcaaaaggtttgtttcaaactgcagaaatacacatatggcatggtattaacatcttttaagtcatggacactgcagcttaccgtgaaaacagacattagaaaggaccagtgaattgtttttgatagagatctgcataagatgagaaggtaatactcatgtcagcttttgaaaatcacacaatctccatgcagaagtcagaccacaaccagcattaaggagcctcatttttaatctggacaactgaaaagtccagatttacaatcaacatttccacaattagaaagaaataaaaagatatagagaaagaagatcttagtgaaacatactgcattccaataggtgcacccaaagaaacagctcccaaagcatttataagagcacctattaaacaaatcaaaacacaggtatagatatagtagacacaatcaagggatactgaatcagcaaaagcacaagatgaaagcataagaagctcagactcactcacatcttcaggtttagcttaacatctagaatttccaggtgaactcattgagcaatcaacttctttgaaaaattagtgagctataataagctgaaaaacagaaatactaaccggcaattagtccgactaagcttcggccaacagctctaaagtactggaaatcaatgagaaaatcatgaatacagagtcaaaacacacaagaccaaaataatactatatgaaaagaaacgatttttttgagcattaagagtgtaaagtgaggatactagcttgttttgtcaatttcataaaagggaaatgagacaagcacaaagcaatcgagttaaaacgagaaattcagtttctttaattctcacagagaacctcagagatgaactatactcaccgagcatttctctgggtttcgtcggaacaagctgtagatgattaccacgatcgggaactcaataatctgaatatcaacatcaaaacaaaaaggctaaaattaactgaaaaatatccactagcaaccaggttatgaaagaaagttttagtacccatagcagacgcagagtgagagttggatcagaaatgagatcgacagagtatttgttacggaccacgtgaaatccgaagatcagaaataacccagtaatcacataaacagcaaaagccccccaagttgatatcgtgatactaacggagatttctggattcttcttctccttcgcctctttcatggcttttcctttctcgtcttcgaaatcacagaacaagtgaagaaagaagacgtaaacaaaatattgaaaatcctccagaacttacactgggccttttattctatatacgggcctacaagtttataccatatgggctttaataggcccatttaattatcaagcggtcgccggagataaaatatatcccggtcggtgaatccagaactctcttgtacgtttgcgcgatttctccacctttccacaatcccctgggttgtgccacgaccttttttctcgaaatgtctcgttcctctcgtcggattcgtatatatagcttcttccatcgtttccgattcttcatcaaacagataaacaaacaaaagaaatcgaaaaacctcacttccaatttcattcaattactgaagcttttttttagca

SEQ ID NO: 6 - DNA, 2340 bp, Artificial Sequence
  HSP18.2pLacIBDGal4ADHSP18.2t
aagcttttctcttcatttctctttctttctttctttcatgcaagttctattgattagaaaccaaaaccatgacatatcacattttcaatttttaaccccaagtttcccaaaaaggtaatcacaattactttccatggtcatttcttctggttcaagcatgacatgaacaggcaataaataagttgagattttgatcacagtaactgatacttgaatcgaatcatttagattttttttttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtcaatggtgaaaccagtaacgttatacgatgtcgcagagtatgccggtgtctcttatcagaccgtttcccgcgtggtgaaccaggccagccacgtttctgcgaaaacgcgggaaaaagtggaagcggcaatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgctatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtatcactggtgaaaagaaaaaccaccctggagccaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtgagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 7 - DNA, 516 bp, Artificial Sequence
  Lac operator (X6 LacI binding site) synthetic
cctaggtacgtatgacttgcgaagatcgatccactagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagctccaccgcggtggcggccggccgctctagtggatcgattccccttcgcaagacccttcctctatataaggaagttcatttcatttggagaggactccggtatttttacaacaattaccacaacaaaacaaacaacaaacaacattacaatttactattctagtcga

SEQ ID NO: 8 - DNA, 2256 bp, Artificial Sequence
  HSP18.2pLacIBDNLSVP64HSP18.2t
aagcttttctcttcatttctctttctttctttctttcatgcaagttctattgattagaaaccaaaaccatgacatatcacattttcaatttttaaccccaagtttcccaaaaaggtaatcacaattactttccatggtcatttcttctggttcaagcatgacatgaacaggcaataaataagttgagattttgatcacagtaactgatacttgaatcgaatcatttagattttttttttttttagtttacttgtttagtaaatatgttgtctatgtttgtcacaaaaacgtggctcagttcttgtatatatggagacaaaaaaatccattaaaagattgttgacattctcggaaatttagtgccaactgttattgcgagaacttactatagttttcctttggcgaaaagctaataatcttaaatcttgattttgtcctcttttctctgagttagattttcttaaattccacttccgacctattaagaaatgggcttttgcaaagaagatccgcttcactgagcccgtatctcgaagaggataatacaacaacaaagcaaaacggcacgtagttttaattgtaaccaaggattgcatttcggtcttgtttcaacaaacgaaacttcctgaaatgccaagaaaaatctggtcatttcaccacagtgatcattgtgtatgtgttctaaagactccaagcgaaggttttagaaaaaggagcattttctattctattcaagaaactcgaagaacattctctcttcatcctctaacttccctataaatatgtcctttgctaatcagatcaaatcagcaggaaaatcaagaaccaaaagtcaatggtgaaaccagtaacgttatacgatgtcgcagagtatgccggtgtctcttatcagaccgtttcccgcgtggtgaaccaggccagccacgtttctgcgaaaacgcgggaaaaagtggaagcggcaatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgctatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtatcactggtgaaaagaaaaaccaccctgcctaagaagaagagaaaggtagacgcattggatgacttcgacttagacatgcttgggtcagatgctttggacgactttgacctcgacatgttgggttctgacgcgctagatgacttcgacttggacatgttgggctccgacgctttggacgactttgatctggacatgttatgagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataag

SEQ ID NO: 9 - DNA, 1935 bp, Antirrhinum majus
atggctactggtatccaaaaccaaaagatagtgcctgagaatttgaggaagcaacttgctattgctgttagaagtatccaatggagttatgcaattttctggtccaattcagttgcacaaccaggggtcttggagtggggtgatgggttctacaatggagatattaaaactcgaaaaactgtacaatctgtcgaattgaatcccgatcagctgggattgcagagaagtgatcaattgagagaactttatgagtctctttcacttggtgaaaccaacacacaagctaaaaggcctactgctgcattatcaccagaagacctcactgatgctgagtggtttttcttggtttgcatgtctttcatattcaatattggccaagggttgcctggaagaacattagcacgaaatcaagcagtatggctatgcaacgctcatcgtgcggacaccaaagttttctcgcgttctttgcttgcaaagagtgcgtcaattcagacagttgtgtgctttccatatgcagaaggagtagttgagctgggagcaacagagctagtaccggaggatttgaatctaatccagcatataaaaacttcattcttggacagtcctgccaccgttcccaagattcccaactatgtctccaacagtattacaaacaacaatgacctcatttgtgaagcgcttgaacatgctaatataccagaaaacgatcttgatcagcttttgaattgtccagacacgaacatatgttctcctgataacagtttggatgactttgcagacaatttactcatagacgaatcgaatttggcagaaggcatcaatggggaggttcctcaaacacaaagctggcctttcatggatgatgcaatcagcaattgtctcaatagttctatgaattctagtgactgtatatctcaaactcatgaaaatctagagtcttttgctccactttctgatggaaaaggggcaccggagacaaataattgtatgcacagcactcaaaaatgcaatcagcagatagaaaacacgggtgtccaaggcgacgaggtccattatcaaggggtactttccaatcttttgaagagttcccatcagttggttcttggtccctacttcagaaatgggaatagagaatcaagcttcgttagttggaacaaggatggatcgtcgggtactcatgttccccgaagcggaacctcacaaagatttctgaagaaagtactttttgaagtagctagaatgcatgaaaactccaggcttgatgctggtaaacaaaaaggcaacagtgactgccttgcaaagccaacggctgatgaaattgatagaaaccacgtcttgtcagagagaaaacgcagagagaaaataaacgaacggtttatgattcttgcatccctagtcccatccggtggcaaggttgacaaagtatcaatactagaccatacaatagattacttgagagggcttgagaggaaagtcgacgagctggaatctaacaaaatggtaaagggccgggggcgggaatcaactacaaaaactaaactgcacgatgccattgagaggacctccgataattatggcacaacaaggacaagtaatgtcaagaaaccgttgacaaacaagagaaaggcttctgatacggacaagattggagccgtaaatagcagaggtcgattgaaagattccttaacagataatataactgtgaacattacaaacaaggatgtgttgattgtcgtgagttgttcttccaaggagtttgtattgcttgaagtgatggaagccgtaagacgactaagtttggattccgaaactgttcaatcttccaacagagatggaattatatccattaccataaaagccaagtgcaagggattgaaggttgcatcagcaagtgtgatcaaacaagctcttcagaaagttactatgaagtcttga

SEQ ID NO: 10 - DNA, 664 bp, Antirrhinum majus
aatggaaaagaattgtcgtggagtgagaaaaggtacttggaccaaagaagaagacactctcttgaggcaatgtatagaagagtatggtgaagggaaatggcatcaagttccacacagagcagggttgaaccggtgtaggaagagttgcaggctgaggtggttgaattatctgaggccaaatatcaaaagaggtcggttttcgagagatgaagtggacctaattgtgaggcttcataagctgttgggtaacaaatggtcgctgattgctggtagaattcctggaaggacagctaatgacgtgaagaacttttggaatactcatgtggggaagaatttaggcgaggatggagaacgatgccggaaaaatgttatgaacacaaaaaccattaagctgactaatatcgtaagaccccgagctcggaccttcaccggattgcacgttacttggccgagagaagtcggaaaaaccgatgaattttcaaatgtccggttaacaactgatgagattccagattgtgagaagcaaacgcaattttacaatgatgttgcgtcgccacaagatgaagttgaagactgcattcagtggtggagtaagttgctagaaacaacggaggatggggaattaggaaacctattcgaggaggcccaacaaattggaaattga

SEQ ID NO: 11 - DNA, 2774 bp, Artificial Sequence
  35SpGRLacIBDGal4ADHSP18.2t
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgaccatggatcctgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatcaatggtgaaaccagtaacgttatacgatgtcgcagagtatgccggtgtctcttatcagaccgtttcccgcgtggtgaaccaggccagccacgtttctgcgaaaacgcgggaaaaagtggaagcggcaatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgctatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtatcactggtgaaaagaaaaaccaccctggagccaacaaattctcaagcgctttcacaaccaattgcctcctctaacgttcatgataacttcatgaataatgaaatcacggctagtaaaattgatgatggtaataattcaaaaccactgtcacctggttggacggaccaaactgcgtataacgcgtttggaatcactacagggatgtttaataccactacaatggatgatgtatataactatctattcgatgatgaagataccccaccaaacccaaaaaaagagtgagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 12 - DNA, 2690 bp, Artificial Sequence
  35SpGRLacIBDNLSVP64HSP18.2t
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactccggtatttttacaacaattaccacaacaaaacaaacaacaaacaacattacaatttactattctagtcgaccatggatcctgaagctcgaaaaacaaagaaaaaaatcaaagggattcagcaagccactgcaggagtctcacaagacacttcggaaaatcctaacaaaacaatagttcctgcagcattaccacagctcacccctaccttggtgtcactgctggaggtgattgaacccgaggtgttgtatgcaggatatgatagctctgttccagattcagcatggagaattatgaccacactcaacatgttaggtgggcgtcaagtgattgcagcagtgaaatgggcaaaggcgataccaggcttcagaaacttacacctggatgaccaaatgaccctgctacagtactcatggatgtttctcatggcatttgccctgggttggagatcatacagacaatcaagtggaaacctgctctgctttgctcctgatctgattattaatgagcagagaatgtctctaccctgcatgtatgaccaatgtaaacacatgctgtttgtctcctctgaattacaaagattgcaggtatcctatgaagagtatctctgtatgaaaaccttactgcttctctcctcagttcctaaggaaggtctgaagagccaagagttatttgatgagattcgaatgacttatatcaaagagctaggaaaagccatcgtcaaaagggaagggaactccagtcagaactggcaacggttttaccaactgacaaagcttctggactccatgcatgaggtggttgagaatctccttacctactgcttccagacatttttggataagaccatgagtattgaattcccagagatgttagctgaaatcatcactaatcagataccaaaatattcaaatggaaatatcaaaaagcttctgtttcatcaaaaatcaatggtgaaaccagtaacgttatacgatgtcgcagagtatgccggtgtctcttatcagaccgtttcccgcgtggtgaaccaggccagccacgtttctgcgaaaacgcgggaaaaagtggaagcggcaatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgctatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtatcactggtgaaaagaaaaaccaccctgcctaagaagaagagaaaggtagacgcattggatgacttcgacttagacatgcttgggtcagatgctttggacgactttgacctcgacatgttgggttctgacgcgctagatgacttcgacttggacatgttgggctccgacgctttggacgactttgatctggacatgttatgagcttcggccatgctagagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgac

SEQ ID NO: 13 - DNA, 1032 bp, Arabidopsis thaliana
atggagaccgatctcaagtcaacctttctcaacgtttattctgttctcaagtctgaccttcttcatgacccttccttcgaattcaccaatgaatctcgtctctgggttgatcggatgctggactacaatgtacgtggagggaaactcaatcggggtctctctgttgttgacagtttcaaacttttgaagcaaggcaatgatttgactgagcaagaggtcttcctctcttgtgctctcggttggtgcattgaatggctccaagcttatttccttgtgcttgatgacattatggataactctgtcacccgccgtggtcaaccttgctggttcagagttcctcaggttggtatggttgccatcaatgatgggattctacttcgcaatcacatccacaggattctcaaaaagcatttccgtgataagccttactatgttgaccttgttgatttgtttaatgaggttgagttgcaaacagcttgtggccagatgatagatttgatcaccacctttgaaggcgaaaaggatttgtccaagtactcattgtcaatccaccgtcgtattgtccagcacaaaacggcttattactcattttatctccctgttgcttgtgcgttgcttatggcgggcgaaaatttggaaaaccatattgacgtgaaaaatgttcttgttgacatgggaatctacttccaagtgcaggatgattatctggattgttttgctgatcccgagacgcttggcaagataggaacagatatagaagatttcaaatgctcttggttggtggttaaggcattagaacgctgcagcgaagaacaaactaagatattatatgagaactatggtaaaaccgacccatcgaacgttgctaaagtgaaggatctctacaaagagctggatcttgagggagtgttcatggagtatgagagcaaaagctacgagaagctgactggagcgattgagggacaccaaagtaaagcaatccaagcagtgctaaaatccttcttggctaagatctacaagaggcagaagtag

SEQ ID NO: 14 - DNA, 3165 bp, Artificial
  Synthetic
atgccgccgctattcaagggactgaaacagatggcaaagccaattgcctatgtttcaagattttcggcgaaacgaccaattcatataatacttttttctctaatcatatccgcattcgcttatctatccgtcattcagtattacttcaatggttggcaactagattcaaatagtgtttttgaaactgctccaaataaagactccaacactctatttcaagaatgttcccattactacagagattcctctctagatggttgggtatcaatcaccgcgcatgaagctagtgagttaccagccccacaccattactatctattaaacctgaacttcaatagtcctaatgaaactgactccattccagaactagctaacacggtttttgagaaagataatacaaaatatattctgcaagaagatctcagtgtttccaaagaaatttcttctactgatggaacgaaatggaggttaagaagtgacagaaaaagtcttttcgacgtaaagacgttagcatattctctctacgatgtattttcagaaaatgtaacccaagcagacccgtttgacgtccttattatggttactgcctacctaatgatgttctacaccatattcggcctcttcaatgacatgaggaagaccgggtcaaatttttggttgagcgcctctacagtggtcaattctgcatcatcacttttcttagcattgtatgtcacccaatgtattctaggcaaagaagtttccgcattaactctttttgaaggtttgcctttcattgtagttgttgttggtttcaagcacaaaatcaagattgcccagtatgccctggagaaatttgaaagagtcggtttatctaaaaggattactaccgatgaaatcgtttttgaatccgtgagcgaagagggtggtcgtttgattcaagaccatttgctttgtatttttgcctttatcggatgctctatgtatgctcaccaattgaagactttgacaaacttctgcatattatcagcatttatcctaatttttgaattgattttaactcctacattttattctgctatcttagcgcttagactggaaatgaatgttatccacagatctactattatcaagcaaacattagaagaagacggtgttgttccatctacagcaagaatcatttctaaagcagaaaagaaatccgtatcttctttcttaaatctcagtgtggttgtcattatcatgaaactctctgtcatactgttgtttgtcttcatcaacttttataactttggtgcaaattgggtcaatgatgccttcaattcattgtacttcgataaggaacgtgtttctctaccagattttattacctcgaatgcctctgaaaactttaaagagcaagctattgttagtgtcaccccattattatattacaaacccattaagtcctaccaacgcattgaggatatggttcttctattgcttcgtaatgtcagtgttgccattcgtgataggttcgtcagtaaattagttctttccgccttagtatgcagtgctgtcatcaatgtgtatttattgaatgctgctagaattcataccagttatactgcagaccaattggtgaaaactgaagtcaccaagaagtcttttactgctcctgtacaaaaggcttctacaccagttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaa

SEQ ID NO: 15 - DNA, 1647 bp, Citrus sinensis
atgtcgtctggagaaacatttcgtcctactgcagatttccatcctagtttatggagaaaccatttcctcaaaggtgcttctgatttcaagacagttgatcatactgcaactcaagaacgacacgaggcactgaaagaagaggtaaggagaatgataacagatgctgaagataagcctgttcagaagttacgcttgattgatgaagtacaacgcctgggggtggcttatcactttgagaaagaaataggagatgcaatacaaaaattatgtccaatctatattgacagtaatagagctgatctccacaccgtttcccttcattttcggttgcttaggcagcaaggaatcaagatttcatgtgatgtgtttgagaagttcaaagatgatgagggtagattcaagtcatcgttgataaacgatgttcaagggatgttaagtttgtacgaggcagcatacatggcagttcgcggagaacatatattagatgaagccattgctttcactaccactcacctgaagtcattggtagctcaggatcatgtaacccctaagcttgcggaacagataaatcatgctttataccgtcctcttcgtaaaaccctaccaagattagaggcgaggtattttatgtccatgatcaattcaacaagtgatcatttatgcaataaaactctgctgaattttgcaaagttagattttaacatattgctagagctgcacaaggaggaactcaatgaattaacaaagtggtggaaagatttagacttcactacaaaactaccttatgcaagagacagattagtggagttatatttttgggatttagggacatacttcgagcctcaatatgcatttgggagaaagataatgacccaattaaattacatattatccatcatagatgatacttatgatgcgtatggtacacttgaagaactcagcctctttactgaagcagttcaaagatggaatattgaggccgtagatatgcttccagaatacatgaaattgatttacaggacactcttagatgcttttaatgaaattgaggaagatatggccaagcaaggaagatcacactgcgtacgttatgcaaaagaggagaatcaaaaagtaattggagcatactctgttcaagccaaatggttcagtgaaggttacgttccaacaattgaggagtatatgcctattgcactaacaagttgtgcttacacattcgtcataacaaattccttccttggcatgggtgattttgcaactaaagaggtttttgaatggatctccaataaccctaaggttgtaaaagcagcatcagttatctgcagactcatggatgacatgcaaggtcatgagtttgagcagaagagaggacatgttgcgtcagctattgaatgttacacgaagcagcatggtgtctctaaggaagaggcaattaaaatgtttgaagaagaagttgcaaatgcatggaaagatattaacgaggagttgatgatgaagccaaccgtcgttgcccgaccactgctcgggacgattcttaatcttgctcgtgcaattgattttatttacaaagaggacgacggctatacgcattcttacctaattaaagatcaaattgcttctgtgctaggagaccacgttccattttga

(19) United States
(12) Patent Application Publication
(10) Pub. No.: US 2024/0279671 A1
(43) Pub. Date: Aug. 22, 2024

(54) PLANTS EXHIBITING A PHENOTYPE UPON A CONTROLLED INDUCTION AND METHODS OF GENERATION THEREOF

(71) Applicant: Pigmentium Ltd, Kiryat Shmona (IL)

(72) Inventors: Tal LUTZKY, Kibbutz Sdot Yam (IL); Amir TYROLER, Hadera (IL); Alexander VAINSTEIN, Rechovot (IL)

(73) Assignee: Pigmentium Ltd, Kiryat Shmona (IL)

(21) Appl. No.: 17/789,200

(22) PCT Filed: Dec. 24, 2020
(86) PCT No.: PCT/IL2020/051333

§ 371 Date: Jun. 26, 2022

(30) Foreign Application Priority Data
Dec. 26, 2019 (IL) 271732

(51) Int. Cl. C12N 15/82

(52) U.S. Cl. CPC C12N 15/825; C12N 15/8238

(57) ABSTRACT

The present disclosure provides a genetic system utilizing recombinant nucleic acid molecule being incorporated in genetic pathways of a plant to allow controllable expression of a certain gene to obtain a desired phenotype expression. The controllable expression is performed by controllably inducing a certain condition at a certain location of the plant that comprises the recombinant nucleic acid molecule. For example, the condition is typically required to be induced locally by application of a certain hormone, exposure to heat and/or light or any other condition to the plant part according to the characteristics of the expression mechanism of the recombinant nucleic acid molecule. Local induction can be obtained by selective physical application of the required condition on the plant part. Local induction may also be obtained by water that carries an inducing agent, e.g. a hormone, and flowing through the watering system of the plant, applied for example by irrigation.

### RELATED APPLICATIONS

This application is a National Phase of PCT Patent Application No. PCT/IL2020/051333 having International filing date of Dec. 24, 2020, which claims the benefit of priority of Israeli Patent Application No. 271732, filed Dec. 26, 2019. The contents of the above applications are all incorporated by reference as if fully set forth herein in their entirety.

### SEQUENCE LISTING STATEMENT

The ASCII file, entitled 92894SequenceListingCorrected.txt, created Sep. 28, 2023, comprising 34,085 bytes, is incorporated herein by reference. The sequence listing submitted herewith is identical to the sequence listing forming part of the international application with correction as to the origin of SEQ ID NO: 13 and 14. This correction is unambiguously derivable from the application. No new matter is added by this correction.

### TECHNOLOGICAL FIELD

The invention relates to the field of genetically modified plants.

### BACKGROUND ART

References considered to be relevant as background to the presently disclosed subject matter are listed below:
[1] Grotewold, E., Chamberlin, M., Snook, M., Siame, B., Butler, L., Swenson, J., Maddock, S., St Clair, G., Bowen, B., Hughes, S., et al. (1998). Engineering secondary metabolism in maize cells by ectopic expression of transcription factors. Plant Cell 10, 721-740.
[2] Schwinn, K., Venail, J., Shang, Y., Mackay, S., Alm, V., Butelli, E., Oyama, R., Bailey, P., Davies, K., and Martin, C. (2006). A small family of MYB-regulatory genes controls floral pigmentation intensity and patterning in the genus Antirrhinum. Plant Cell 18, 831-851.
[3] Ben-Zvi, M. M., Negre-Zakharov, F., Masci, T., Ovadis, M., Shklarman, E., Ben-Meir, H., Tzfira, T., Dudareva, N., and Vainstein, A. (2008). Interlinking showy traits: co-engineering of scent and colour biosynthesis in flowers. Plant Biotechnol. J. 6, 403-415.
[4] Moore, I., Samalova, M., and Kurup, S. (2006). Transactivated and chemically inducible gene expression in plants. Plant J. 45, 651-683.
[5] Beerli, R. R., Segal, D. J., Dreier, B., and Barbas, C. F. (1998). Toward controlling gene expression at will: Specific regulation of the erbB-2/HER-2 promoter by using polydactyl zinc finger proteins constructed from modular building blocks. Proc. Natl. Acad. Sci. 95.
[6] Leitner-Dagan Y., Ovadis M., Shklarman E., Elad Y., Rav David D., Vainstein A. (2006). Expression and functional analyses of the plastid lipid-associated protein CHRC suggest its role in chromoplastogenesis and stress. Plant Physiol. 142(1):233-244.
[7] Yoshida, K, Kasai, T., Garcia, M. R. C., Sawada S., Shoji T, Shimizu S., Yamazaki K., Komeda Y., Shinmyo T. (1995) Heat-inducible expression system for a foreign gene in cultured tobacco cells using the HSP18.2 promoter of Arabidopsis thaliana. Appl Microbiol Biotechnol. 44, 466-472.
[8] Matz, M., Fradkov, A., Labas, Y. et al. Fluorescent proteins from nonbioluminescent Anthozoa species. Nat Biotechnol 17, 969-973 (1999).

Acknowledgement of the above references herein is not to be inferred as meaning that these are in any way relevant to the patentability of the presently disclosed subject matter.

### BACKGROUND OF THE INVENTION

It is known that overexpression of LC and C1 genes originating from corn, PAP1 originating from Arabidopsis and the Rosea and Delila genes results in accumulation of anthocyanin in different tissues of plants [1-3].

### SUMMARY OF THE INVENTION

The present disclosure provides a genetic system utilizing recombinant nucleic acid molecule being incorporated in genetic pathways of a plant to allow controllable expression of a certain gene to obtain a desired phenotype expression. The controllable expression is performed by controllably inducing a certain condition at a certain location of the plant that comprises the recombinant nucleic acid molecule. For example, the condition is typically required to be induced locally by application of a certain hormone, exposure to heat and/or light or any other condition to the plant part according to the characteristics of the expression mechanism of the recombinant nucleic acid molecule. Local induction can be obtained by selective physical application of the required condition on the plant part. Local induction may also be obtained by water that carries an inducing agent, e.g. a hormone, and flowing through the watering system of the plant, applied for example by irrigation. Every plant part that is being contacted with the hormone in the water expresses the desired gene that yields the desired phenotype. The phenotype may be an expression of any one of: nucleic acid molecules (DNA and/or RNA) proteins (e.g. enzymes involved in biosynthetic pathways), pigments (e.g. anthocyanins), metabolites, terpenes, or any other outcome of a genetic expression.

It is to be noted that the genetic system of the present disclosure may result in expression of any desired protein or other compound that is the result of the genetic pathway that is induced by the genetic system.

The induction system of the present disclosure can be used to controllably induce genes of edible plants or crops that result in expression of sugars, proteins, vitamins, antioxidants, or any other substances that may improve the quality of the crop or the plant, either with respect to its taste or its valuable nutritional values. For example, lettuce that is fully grown, prior to its harvest it can be controllably induced to produce more protein and red pigment.

Thus, an aspect of the present disclosure provides a plant comprising a recombinant nucleic acid molecule that encodes for at least one gene. The recombinant nucleic acid molecule expression is activated upon induction of a certain condition, e.g. binding of a certain hormone or application of heat that results in a desired phenotypic expression, e.g. production of anthocyanins, a metabolite, a protein or any other molecule. The induction of expression may be performed at any stage of the plant's development. In certain embodiments the induction is performed when the plant is fully grown and reached a high biomass thereby inducing a high expression level of the recombinant nucleic acid molecule, and/or any protein encoded by the recombinant nucleic acid, and/or any product produced directly or indirectly by the expression of the recombinant nucleic acid molecule.

In a first of its aspects the present invention therefore provides a genetically modified plant, comprising a recombinant nucleic acid molecule that encodes for at least one gene, wherein upon induction of expression of said gene in one or more parts thereof, said genetically modified plant exhibits a visible phenotypic change at a predesigned location.

As used herein, the term “predesigned location” refers to the specific location where induction was performed, e.g. by locally applying heat or locally administering a hormone, such that other parts of the plant remain unchanged.

In certain embodiments, the predesigned location is the whole plant.

In one embodiment, the phenotypic change is a color change or luminosity. In some other embodiments, the phenotypic change or expression is production of a certain product (e.g. a compound) in the plant. The phenotypic change may be a result of expression in the plant part of any one of the group consisting of: flavonoids such as: anthocyanidins, flavanols, flavones, flavonols, flavonones and isoflavones, betalains, chromoproteins, Terpenes: e.g. monoterpenes (genipin, limonene) (C₁₀), sesquiterpenes (valencene, artimesnin) (C₁₅), diterpenes (retinol, retinal, Taxol, and phytol) (C₂₀), Sesterterpenes (Rebaudioside A, Merochlorin A, Ophiobolin A, Ophiobolin B, Ophiobolin C) (C₂₅), Triterpenes (Mogrosides, Squalene, α-Amyrin, β-Amyrin, Cucurbitacin B, Sitosterol, Stigmasterol, Campesterol, α-Spinasterol) (C₃₀), Carotenoids (Phytofluene, Lycopene, Cynthiaxanthin, Pectenoxanthin, Lutein, Zeaxanthin, Glycosides, Astaxanthin) (C₄₀), Alkaloids such as: Ajmalicine, Ajmaline, Berberine, Camptothecin, Capsaicin, Capsorubin, Codeine, Colchicine, Ellipticine, Emetine, Morphine, Quinine, Sanguinarine, Vincristine, Vinblastine. proteins such as: albumin, globulin, glutelin, Melatonin, and any combination thereof.

As used herein, the term “color change” refers to a modification of the pattern of expression of pigments and the term “bioluminescence activity” refers to a bioluminescence activity such as the expression of a luciferase protein for example which catalyzes luciferin, or expression of fluorescent proteins resulting in emission of blue-green, red or yellow light (8).

In one embodiment, the color change or luminosity form a predesigned colored pattern.

As used herein, the term “predesigned colored pattern” refers to a particular, desired or tailor-made shape for example such a shape may represent a particular logo or trademark, a particular shape (e.g. a heart, as demonstrated in FIG. 5C) or a shape in relation with a particular event (e.g. a figure of Santa Klaus for Christmas event), letters, spots and the like.

Said visible phenotypic change may be stable or transient.

In certain embodiments, said at least one gene is a regulatory gene, a gene encoding a protein involved in plant pigmentation, a chromoprotein encoding gene, a gene encoding a protein involved in bioluminescence, a gene encoding an enzyme involved in the production of a compound selected from a group consisting of: flavonoids (e.g. anthocyanidins, flavanols, flavones, flavonols, flavonones or isoflavones), betalains, chromoproteins, Terpenes (e.g. monoterpenes (genipin, limonene) (C₁₀), sesquiterpenes (valencene, artimesnin) (C₁₅), diterpenes (retinol, retinal, Taxol, and phytol) (C₂₀), Sesterterpenes (Rebaudioside A, Merochlorin A, Ophiobolin A, Ophiobolin B, Ophiobolin C) (C₂₅), Triterpenes (Mogrosides, Squalene, α-Amyrin, β-Amyrin, Cucurbitacin B, Sitosterol, Stigmasterol, Campesterol, α-Spinasterol) (C₃₀), Carotenoids (Phytofluene, Lycopene, Cynthiaxanthin, Pectenoxanthin, Lutein, Zeaxanthin, Glycosides, Astaxanthin) (C₄₀), Alkaloids (e.g. Ajmalicine, Ajmaline, Berberine, Camptothecin, Capsaicin, Capsorubin, Codeine, Colchicine, Ellipticine, Emetine, Morphine, Quinine, Sanguinarine, Vincristine, Vinblastine), or a gene encoding a protein (e.g. albumin, globulin, glutelin, Melatonin).

In one embodiment, said recombinant nucleic acid molecule further comprises a binding site for a transcription factor of said gene.

In certain embodiments, said binding site is a promoter or an enhancer.

In one embodiment, the induction of expression of said at least one gene is performed by local activation. In certain embodiments the activation is performed by subjecting the plant to at least one external condition which is not required for the plant's regular growth. In other words, the regular growth of the plant is not affected until a specific external condition is applied, which triggers the gene pathway expression that results in the specific desired phenotypic expression.

In certain embodiments, the activation is being selected from high temperature, exposure to light characterized by a certain wavelength, injuring, application of response inducing material or a combination thereof.

As used herein, the term “high temperature” refers to a temperature ranging from 35° C.-65° C. In some specific embodiments, said temperature is preferably about 50° C.

In certain embodiments, the activation may be performed by using a brush or a laser.

In certain embodiments, said response inducing material is selected from the group consisting of Gibberellin hormone, dexamethasone, abscisic acid (ABA), β-Aminobutyric Acid (BABA), ethanol, auxin, cytokinin (CK), strigolactone, salicylic acid, protocatechuic acid (PCA), Vanilic acid (VA), phloretin and any combination thereof.

In certain embodiments, the parts exhibiting the phenotypic change are being selected from: leaves, stem, flowers, roots, and branches, and any combination thereof.

In certain embodiments, the phenotypic change is due to expression in the plant part of any one of the group consisting of: flavonoids (e.g. anthocyanidins, flavanols, flavones, flavonols, flavonones or isoflavones), betalains, chromoproteins, Terpenes (e.g. monoterpenes (genipin, limonene) (C₁₀), sesquiterpenes (valencene, artimesnin) (C₁₅), diterpenes (retinol, retinal, Taxol, and phytol) (C₂₀), Sesterterpenes (Rebaudioside A, Merochlorin A, Ophiobolin A, Ophiobolin B, Ophiobolin C) (C₂₅), Triterpenes (Mogrosides, Squalene, α-Amyrin, β-Amyrin, Cucurbitacin B, Sitosterol, Stigmasterol, Campesterol, α-Spinasterol) (C₃₀), Carotenoids (Phytofluene, Lycopene, Cynthiaxanthin, Pectenoxanthin, Lutein, Zeaxanthin, Glycosides, Astaxanthin) (C₄₀), Alkaloids (e.g. Ajmalicine, Ajmaline, Berberine, Camptothecin, Capsaicin, Capsorubin, Codeine, Colchicine, Ellipticine, Emetine, Morphine, Quinine, Sanguinarine, Vincristine, Vinblastine), proteins (e.g. albumin, globulin, glutelin, Melatonin) and any combination thereof.

In one embodiment, said at least one gene is at least one regulatory gene involved in anthocyanin gene expression.

In certain embodiments, said at least one gene is selected from a group consisting of the LC gene from maize, the C1 gene from maize, the PAP1 gene from Arabidopsis, the Rosea gene from Antirrhinum, the Delila gene from Antirrhinum, flavanone 3β-hydroxylase (F3H), any combination thereof or any homologous gene thereof.

In one embodiment, said at least one gene consists of the PAP1 gene, the Rosea gene and the Delila gene.

In some embodiments, said at least one gene is selected from a group consisting of HMG-R gene from yeast, FPPS gene from Arabidopsis, TPS gene from Citrus, any homologous gene thereof or any combination thereof. These genes can be used to trigger Valencene biosynthesis.

In some embodiments, the homology of the homologous gene is at least 70%, at least 75%, at least 80%, at least 85%, at least 90%, at least 95%, at least 96%, at least 97%, at least 98% or at least 99%.

In one embodiment, said gene is a gene encoding a protein involved in plant pigmentation, and at least one endogenous gene responsible for pigmentation in said plant is knocked-out.

In one embodiment, the at least one endogenous gene responsible for pigmentation is knocked-out using CRISPR.

In one embodiment, said endogenous gene responsible for pigmentation is knocked-out due to a naturally occurring mutation.

As used herein, the term “naturally occurring mutation” relates to a mutation which was present in the plant without artificial involvement e.g. a plant in which an endogenous gene responsible for pigmentation is knocked-out due to a naturally occurring mutation grows without exhibiting pigmentation and according to the invention, upon induction, a particular gene involved in pigmentation will be expressed.

In certain embodiments, said plant is selected from the group consisting of tobacco plants, Tabacum, Lettuce, Tomato, Corn, Carrot, Soy, Peppers, Gerbera, Petunia, Euphorbia pulcherrima, Kalanchoe, Sansevieria, Grass, Aspidistra, Arabidopsis thaliana, Cabbage, Rosemary, Sage, Potato, Geranium and Solidago.

In one embodiment, the induction of expression of said gene is performed when the plant is fully grown.

In one embodiment, the compound that is produced as a result of the induced expression of said gene is being collected from said plant 1 day, or 2 days, or 3 days, or 4 days, or 5 days or 6 days or more after induction of the expression of said gene.

In one embodiment, the concentration of said compound is between about 0.1%, 0.2%, 0.3%, 0.4%, 0.5%, 0.6%, 0.7%, 0.8%, 0.9%, 1.0% and about 2%, 2.1%, 2.2%, 2.3%, 2.4%, 2.5%, 2.6%, 2.7%, 2.8%, 2.9%, 3%. In some embodiments, the concentration of said compound is about 1.75%.

In one embodiment, the produced compound is collected from said plant by extraction.

It is to be noted that the extraction can be carried out by any known method in the art.

In a specific embodiment, the collection of the compound from the plant is performed by the following process: the plant part is being dried (e.g. using liquid nitrogen), crushed, and subjected to a single or a double extraction, for example using acidic ethanol at 40° C., the solid parts may then be separated by centrifugation [please provide additional options for extracting the compound].

In one embodiment, the genetically modified plant is a transgenic plant.

As used herein, the term “transgenic plant” refers to a plant that encodes a heterologous DNA sequence, or one or more additional DNA sequences that are not naturally endogenous to the plant (collectively referred to herein as “transgenes”), and is chromosomally integrated into the genome of the plant. As a result of such transfer and integration, the transgenic sequence may be transmitted to the next generation of plants.

The nucleic acid molecule comprises one or more inducible promoters, that are selected according to the desired gene activation mechanism. For example, the promoter may be selected so that the activation mechanism is by application of hormone and/or heat exposure above a certain threshold for a predetermined time. Furthermore, the nucleic acid molecule comprises one or more transcription factors' binding sites.

It is to be noted that the recombinant nucleic acid molecule may include any desired gene to be expressed. In other words, the activation and expression mechanism that are located upstream and/or downstream the gene to be expressed, namely the promoter, the transcription factors' binding sites and/or terminators sequences, can be combined with any gene pathway to be expressed to obtain the desired controlled gene expression.

In some embodiments, the activation of the gene expression is triggered by high temperature, exposure to light characterized by a certain wavelength, injuring, application of response inducing material or any combination thereof.

In some embodiments, the gene to be expressed is a regulatory gene of a gene pathway of the plant. The regulatory gene expression is required for expression of an additional gene that results in the desired phenotypic expression.

It is to be noted that the induction can be performed on parts of the plant that are cut from the main plant, namely parts or tissues that have been separated from the main plant, e.g. leaves or branches or any other remains of the plant that are left after its harvest. The induction can also be performed on an unrooted plant, as long as the genetic pathways are viable and inducible.

In another aspect, the present invention provides a reproductive or propagation material of the genetically modified plant of the invention.

In one embodiment, said reproductive or propagation material is a shoot or a seed.

In another aspect, the present invention provides a method for generating a genetically modified plant comprising:
(a) transforming a plant cell in a plant tissue or plant part with a recombinant nucleic acid molecule encoding at least one gene, wherein said at least one gene is capable of affecting a phenotypic change in a plant part; and wherein upon induction of expression of said gene, said genetically modified plant exhibits a visible phenotypic change at the location of the induction; and
(b) regenerating a transformed plant from said plant cell.

In certain embodiments, said plant is selected from the group consisting of tobacco plants, Gerbera, Petunia, Tabacum, Euphorbia pulcherrima, Kalanchoe, Grass, Aspidistra, Arabidopsis thaliana, and Solidago.

In certain embodiments, said plant tissue or plant part is a leaf disc, root, hypocotyl, epicotyl, stem, petal, cotyledon, callus, embryo, seed, or shoot-apical-meristem.

In certain embodiments, said transforming step is performed by any one of contacting said plant tissue or plant part with an agrobacterium carrying at least one recombinant inducible nucleic acid molecule encoding the at least one gene or at least one inducible vector comprising said recombinant nucleic acid molecule, or DNA bombardment of said plant tissue or plant part with a gene gun, or meristem injection.

As used herein, the term “inducible” refers to a gene that is controlled by an inducible promoter. An “inducible promoter” refers to a regulatory region that is operably linked to one or more genes, wherein expression of the gene(s) is increased in the presence of an inducer of said regulatory region. An “inducible promoter” refers to a promoter that initiates increased levels of transcription of the coding sequence or gene under its control in response to a stimulus or an exogenous environmental condition.

In one embodiment, said recombinant nucleic acid molecule or said vector is introduced into the agrobacterium by electroporation.

In certain embodiments, said at least one vector is selected from a group consisting of a plasmid, a viral vector and a bacterial vector.

In one embodiment, said plasmid is a binary plasmid.

In certain embodiments, said gene is a regulatory gene, a gene encoding a protein involved in plant pigmentation, a chromoprotein encoding gene, or a gene encoding a protein involved in bioluminescence.

In one embodiment, said at least one gene is a regulatory gene involved in anthocyanin gene expression.

In certain embodiments, said at least one gene is selected from a group consisting of the LC gene or the C1 gene from maize, the PAP1 gene from Arabidopsis, and the Rosea gene or the Delila gene from Antirrhinum, flavanone 3β-hydroxylase (F3H), petunia AN1, AN2, AN1, Tomato Myb12, Vitis vinifera and any combination thereof.

In one embodiment, said at least one gene consists of the PAP1 gene, the Rosea gene and the Delila gene

In one embodiment, said transforming step is performed by transfecting an agrobacterium with at least one inducible plasmid, wherein said at least one inducible plasmid comprises (i) a promoter operatively linked to a nucleic acid sequence encoding a synthetic transcription protein comprising a DNA binding domain and a transcription activating domain, and (ii) multiple operator sequences operatively linked to at least one promoter said promoter being further operatively linked to at least one gene.

In one embodiment, said plasmid further comprises a sequence encoding NeoR/KanR.

In one embodiment, said plasmid further comprises a sequence encoding glucocorticoid receptor (GR).

In certain embodiments, said at least one promoter is selected from the group consisting of the carotenoid associated gene (CHRC) promoter, a truncated CHRC promoter, heat shock protein (HSP) 18.2 promoter, HSP 70B promoter, the cauliflower mosaic virus (CaMV) 35S promoter and minimal CaMV 35S promoter.

In one embodiment, said DNA binding domain is LacIBD.

In one embodiment, the operator is lac operator.

In one embodiment, said transcription activating domain is Gal4AD.

In one embodiment, said transcription activating domain is VP64 optionally linked to a nucleus localization sequence (NLS).

In one embodiment, said plasmid comprises the PAP1 gene and the Delila gene.

In another embodiment, said plasmid comprises the Rosea gene and the Delila gene.

In yet another embodiment, said plasmid comprises the PAP1 gene, the Rosea gene and the Delila gene.

In one embodiment, the plasmid comprises a CaMV35 promoter operatively linked to a nucleic acid sequence encoding a synthetic transcription protein comprising LacIBD binding domain and the transcription activating domain Gal4AD, operatively linked to the CaMV 35S terminator, and further comprising sequentially multiple sequences of the lac operator operatively linked to CaMV 35S minimal promoter, further operatively linked to the Pap1 gene, the Delila gene and the Rosea gene.

In one specific embodiment, said multiple sequences of the lac operator consist of 6 repeats of the lac operator.

In one embodiment, at least one endogenous gene responsible for pigmentation in said plant is knocked-out and said recombinant nucleic acid comprises at least one gene encoding a protein involved in plant pigmentation.

In certain embodiments, said at least one gene encoding a protein involved in plant pigmentation is selected from the group consisting of anthocyanins, flavonoids, carotenoids, betalains, chromoproteins, and any combination thereof.

In one embodiment, said transforming step is performed by transfecting an agrobacterium with at least one inducible plasmid, wherein said at least one inducible plasmid comprises at least one gene encoding a protein involved in plant pigmentation and at least one regulatory gene.

In one embodiment, said inducible plasmid comprises the genes encoding PhF3H, the Rosea gene and the Delila gene.

In one embodiment, said inducible plasmid further comprises at least one of: a Solanum lycopersicum ubiquitin promoter (SlPrUbiq), a BASTA resistance gene (BlpR), a Solanum lycopersicum terminator (TeUbiq), an HSP18.2 promoter and an HSP18.2 terminator flanking the PhF3H gene, and a CaMV 35S promoter and a NOS terminator flanking each of the Rosea and the Delila genes.

In one embodiment, said inducible plasmid further comprises LacIBD, Gal4AD, multiple sequences of lac operator, and minimal CaMV 35S promoter.

In certain embodiments, said recombinant nucleic acid molecule encoding at least one gene comprises SEQ ID NO: 2, SEQ ID NO: 9 or SEQ ID NO: 10.

In another aspect, the present invention provides a method for generating the genetically modified plant of the invention, said method comprising:
(a) transforming a plant cell in a plant tissue or plant part with a nucleic acid molecule encoding an heterologous gene, wherein said heterologous gene is capable of affecting a phenotypic change in a plant part;
(b) regenerating a transformed plant from said plant cell; and
(c) inducing expression of said heterologous gene on one or more plant parts of said transformed plant; wherein upon induction of said heterologous gene expression, said genetically modified plant exhibits a visible phenotypic change at the location of the induction.

In certain embodiments, said induction of local expression is performed by a means selected from a group consisting of: locally exposing said genetically modified plants to a high temperature, exposure to light characterized by a certain wavelength, injuring, application of response inducing material or a combination thereof.

In certain embodiments, said high temperature ranges from between about 35° C.-65° C., preferably about 50° C.

In one embodiment, said at least one gene is a gene involved in anthocyanin gene expression.

In one embodiment, the method further comprises knocking out the endogenous gene responsible for anthocyanin expression in said genetically modified plant prior to step (a).

In one embodiment, said knocking-out is performed using CRISPR.

In another aspect, the present invention provides an agrobacterium comprising at least one plasmid as described above.

In another aspect, the present invention provides a plant cell comprising at least one plasmid as described above.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1: Low expression levels of the PAP1 gene in HSP:PAP1 and CHRC:PAP1 following induction.

Lines from HSP:PAP1 and CHRC:PAP1 transgenic plants were induced using heat and injury respectively. RNA was isolated from each of the lines and the expression level of PAP1 was examined in these plants as well as wild type plants and 35S:PAP1 plants.

FIG. 2: Schematic representation of the plasmid system for transactivation.

These plasmids were constructed based on LacI in combination with Gal4 or VP64 in order to allow efficient induction of the target gene in response to a heat shock.

FIG. 3A-3B: GUS activity in leaves of transgenic plants.

FIG. 3A: GUS activity in leaves of a transgenic plant including the construct HSP18.2:LacIBD:Gal4AD/pOp:GUS without heat shock (upper line) and following induction by heat shock (lower line).

FIG. 3B: GUS activity in leaves of a transgenic plant including the construct HSP18.2:LacIBD:NLS:VP64 without heat shock (upper line) and following induction by heat shock (lower line).

FIG. 4A-4E: Production of transgenic plants with constructs including pOp:PAP1.

FIG. 4A: Callus accumulating anthocyanins in leaves transformed with the system HSP18.2:LacIBD:NLS:VP64/pOp:PAP1.

FIG. 4B: Callus accumulating anthocyanins in leaves transformed with the system HSP18.2:LacIBD:Gal4AD/pOp:PAP.

FIG. 4C: Callus accumulating anthocyanins in leaves transformed with the system 35S:LacIBD:NLS:VP64/pOp:PAP.

FIG. 4D: Callus accumulating anthocyanins in leaves transformed with the system 35S:LacIBD:Gal4AD/pOp:PAP1.

FIG. 4E: Young transgenic plants including the construct 35S:LacIBD:Gal4AD/pOp:PAP1 following one month of tissue culture.

FIG. 5A-5C: Production of pigments in Tobacco leaves as a result of the expression of the genes 35S:PAP1, 35S:Rosea, and 35S:Delila.

FIG. 5A: Examples of leaves expressing different combinations of the genes Rosea, Delila and PAP1.

FIG. 5B: Heart pattern that was obtained in leaves of Tobacco expressing the three genes together.

FIG. 5C: Pattern that was obtained in leaves of Tobacco expressing the three genes together.

FIG. 6A-6B: Accumulation of pigments as a results of induction in leaves expressing 35S:Delila, 35S:Rosea and HSP18.2:LacBD:Gal4AD/pOp:PAP1.

FIG. 6A: Leaves without induction.

FIG. 6B: Leaves following induction by a heat shock.

FIG. 7A-7C: Schematic representation of the plasmid systems for inducible activation of anthocyanin production genes

FIG. 7A: Transactivation of regulatory genes responsible for anthocyanin production, based on induction of a synthetic transcription activator by Dexamethasone.

FIG. 7B: Transactivation of the anthocyanin biosynthetic gene F3H, based on induction of a synthetic transcription activator by heat shock.

FIG. 7C: Direct induction for the anthocyanin biosynthetic gene F3H by heat shock.

FIG. 8: Transient expression of 35S:Rosea/35S:Delila and 35S:F3H in a leaf of tobacco f3h mutant leads to anthocyanin accumulation only in the presence of correct F3H gene.

FIG. 9: is a graph showing anthocyanin expression (as % anthocyanin/fresh weight) in tobacco leaves of a wild-type (WT) plant, a modified plant that was not exposed to the inducing agent (not induced), a plant subjected to overexpression, and in the modified plant 3, 4 and 5 days post induction.

FIG. 10: is a photograph of a tobacco leaf of the modified plant 5 days following induction.

### DETAILED DESCRIPTION OF THE INVENTION

The present inventors have shown that locally inducing the expression of particular target genes associated with genetical pathways of production of desired compounds or organic matter can result in a desired phenotypic expression. For example, the expression of genes associated with plant pigmentation can be used to generate a desired graphical pattern on plant leaves.

In another example, the expression of genes associated with the production of anthocyanins can result in accumulation of anthocyanins in the plant that can be collected, e.g. by known extraction methods, for further industrial use. It is to be noted that by using the technique of the present disclosure, the yield of the accumulation of anthocyanins that can be obtained (e.g. in the leaves or any other part of the plant) is significantly higher than by using known methods of overexpression.

Furthermore, the technique of the present disclosure can be used to express cytotoxic, volatile or any other non-stable materials that can be relatively easily be collected following their expression. Since the technique of the present disclosure is induction-based technique, the expression of such materials can be controlled and timed to yield the desired compound production and/or accumulation.

In plants amenable to genetic engineering, inducible heterologous systems are an elegant and efficient approach to overcome spatial and developmental limitations for production of specialized metabolites.

Accordingly, in one of its aspects the present invention provides a genetically modified plant, comprising a recombinant nucleic acid molecule that encodes for at least one gene, wherein upon induction of expression of said gene in one or more parts thereof, said genetically modified plant exhibits a phenotypic change at a predesigned location.

In some embodiments, the phenotypic change is visible and remains stable overtime, thereby creating a plant which stably displays a desired graphical pattern. This predesigned graphical pattern can be in the form of letters, shapes, spots, lines and the like. The final displayed pattern is generated only at the time of the external induction, and only at the specific location of the induction, such that other parts of the plant remain unchanged.

In some embodiments, the phenotypic change appears on the genetically modified plant of the invention is transient. In such case, the pattern will diminish with time.

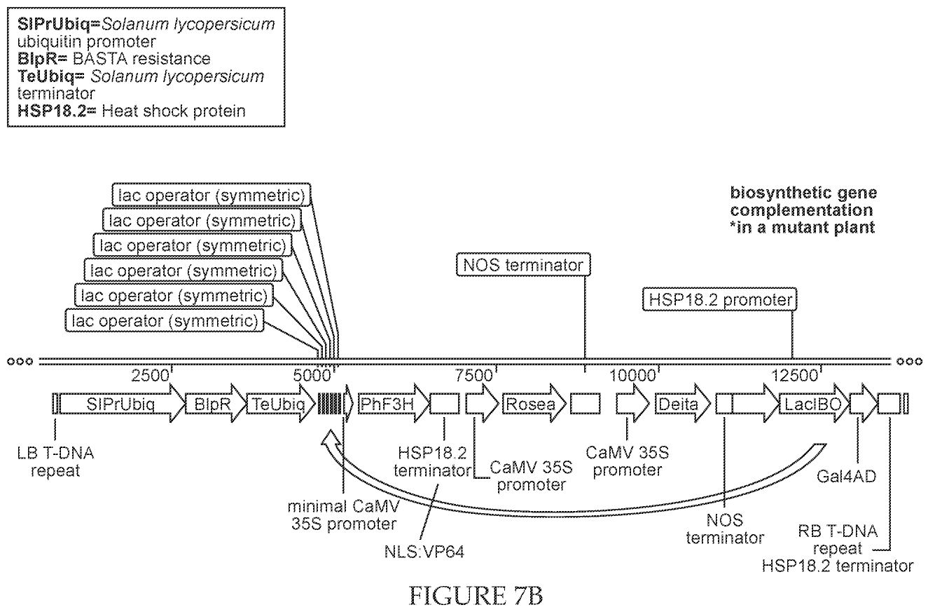

An example of an inducible plasmid of the invention is schematically represented in FIG. 7C.

A recombinant nucleic acid molecule that encodes for at least one gene, the expression of said recombinant nucleic acid molecule is activated upon induction of a certain local condition that results in a desired phenotypic expression.

In certain embodiments, the recombinant nucleic acid molecule further comprising:

one or more inducible promoters; one or more transcription factors' binding sites. The one or more inducible promoters are selected according to the desired gene activation mechanism. The activation mechanism is selected from high temperature, exposure to light characterized by a certain wavelength, injuring, application of response inducing material or any combination thereof.

In certain embodiments of the recombinant nucleic acid molecule, the response inducing material is selected from the group consisting of Gibberellin hormone, dexamethasone, abscisic acid (ABA), β-Aminobutyric Acid (BABA), ethanol, auxin, cytokinin (CK), strigolactone, salicylic acid, Protocatechuic acid (PCA), Vanilic acid (VA), Phloretin or any combination thereof.

In certain embodiments of the recombinant nucleic acid molecule, said one or more transcription factors' binding sites are selected from yeast or E. coli.

In certain embodiments of the recombinant nucleic acid molecule, said at least one gene is at least one regulatory gene involved in anthocyanin gene expression.

In certain embodiments of the recombinant nucleic acid molecule, said at least one gene is selected from a group consisting of the LC gene from maize, the C1 gene from maize, the PAP1 gene from Arabidopsis, the Rosea gene from Antirrhinum, the Delila gene from Antirrhinum, flavanone 3β-hydroxylase (F3H) or any combination thereof.

In certain embodiments of the recombinant nucleic acid molecule, said at least one gene consists of the PAP1 gene, the Rosea gene and the Delila gene.

In some embodiments of the recombinant nucleic acid molecule, said at least one gene is selected from a group consisting of HMG-R gene from yeast, FPPS from Arabidopsis, TPS from Citrus or any combination thereof. These genes can be used to trigger Valencene biosynthesis.

In some embodiments of the recombinant nucleic acid molecule, said gene is a gene encoding a protein involved in plant pigmentation, and wherein at least one endogenous gene responsible for pigmentation in said plant is knocked-out.

In some embodiments, the recombinant nucleic acid molecule is comprised in a plant.

All definitions, as defined and used herein, should be understood to control over dictionary definitions, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

Transformation and Regeneration of Tobacco (Nicotiana tabacum) from Leaf Discs with Recombinant Agrobacterium 2011

Tobacco plants are kept in boxes in sterile conditions in a growth room.

MS regeneration medium (1 liter): 4.4 gr MS salts with vit (Sigma M5519), or 4.71 gr (Duchefa M0222), 2% sucrose, 1% Manitol, 0.8% bacto agar (pH 5.8). Autoclave. Cool down to about 55° C. and add IAA (0.1 mg/L), Zeatin (2 mg/L).

### Transformation

1. Grow Agrobacterium carrying Binary plasmid contains X gene in 20 ml of LB with proper ab (50 mg/l kanamycin) and 50 μg/ml of Rifampicin. Add acetosyringone to a final concentration of 50 μM over-night at 28° C. with shaking (200 rpm). Until OD₆₀₀ is 0.6.
2. Centrifuge for 10 minutes at 4000 g.
3. Resuspend the pellet with liquid MS 2% glucose. Add acetosyringone to a final concentration of 50 μM.
4. Transfer the liquid of the bacteria to Petri dishes.
5. Cut Sterilization leaf discs (1-1.5 cm in diameter) on sterile Whatmann filter paper 3 MM
6. Incubate the leaf disc with the agro. for 10 min
7. Dry on sterile Whatmann 3 MM
8. Place the infected leaf disc (adaxial side down) on Petri dishes with MS regeneration medium.
9. Incubate the infected leaf disc on Petri dishes for 48 h in the dark.
10. Transfer the leaf discs to fresh MS regeneration medium supplemented with: 300 μg/ml carbenicillin, (150-200 μg/ml kanamycin,) for selection.
11. Transfer to fresh medium every 10 days.
12. Incubate the plates 2-3 weeks in growth room (same condition as before) until shoots appear
13. Excise the shoots and transfer them to rooting medium: MS 2% sucrose, 0.8% bacto agar (pH 5.8) with 250 mg/l carbenicillin and 150 mg/l kanamycin, for further elongation.

### Hardening

1—Hardening of the plants is performed as follows:
2—Well-developed plantlets are transferred into a pot containing potting soil and are covered with a plastic bag. The pots are placed in the greenhouse.
3—After 3 days, the bags are excised at their top to produce holes that allow air exchange. The holes can be used to irrigate the plantlets as well.
4—After about 3-4 days, the plastic bag is removed.
5—The plantlets can be propagated by cuttings.

### Example 1

In order to activate the anthocyanin pathway in leaves, sequences encoding several regulatory genes belonging to this pathway were isolated, including LC and C1 genes originating from corn, PAP1 originating from Arabidopsis and the Rosea and Delila genes from Antirrhinum. It is known that overexpression of these genes results in accumulation of anthocyanin in different tissues. The isolated sequences were inserted into plasmids, sequenced and fused to several promoters and terminators thereafter.

In order to examine whether the activation of the anthocyanin pathway may be inducible, the sequence encoding PAP1 was fused to several promoters that respond to environmental factors or chemical substances: CHRC promoter that induces gene expression in response to a cut or injury and detection of Gibberellin hormone; a fragment of the CHRC promotor that induces gene expression in response to a cut or injury only; and the promoters HSP18.2 and HSP70B that induce gene expression following an exposure to high temperatures for a predetermined period of time, i.e. a heat shock.

The genes that were prepared under the promoters as described above, were inserted into binary plasmids. The plasmids were transformed by electroporation to agrobacterium suitable for transformation into plants. Transformation into tobacco leaves was performed and following a one-month selection in the presence of antibiotics, transgenic plants were formed in culture. The transgenic plants were examined at the DNA level for the presence of the insert using PCR, and the plants that were identified as having the insert, were moved to grow in a greenhouse.

A detailed description of these binary plasmids is provided in Table 1.

TABLE 1
The genes that were constructed under the promoters as detailed above, were incorporated
into binary plasmids. The plasmids were introduced by electroporation into agrobacterium
suitable for plant transformation. Transformation was performed in Tobacco leaves.
Following one month of selection with antibiotic, transgenic young plants were produced
in culture. The transgenic plants were examined at the DNA level for presence of the
transgene by using PCR, and the genes that contained the exogenous genes were put outside
the culture for growth in a greenhouse.

| | | | | Promoter | |
|---|---|---|---|---|---|
| | | | | sequence/ | |
| Inducible | Transcription | Activation | Anthocyanin | transcription | |
| promoter | factor (gene) | signal | accumulation | activator | Gene sequence |
| CHRC | PAP1 | Cut and | No | CHRC(1460)p | PAP1 |
| promoter | | sensing | observation | as denoted by | as denoted by |
| | | Gibberellin | | SEQ ID NO: 1 | SEQ ID NO: 2 |
| | | hormone | | | |
| Truncated | PAP1 | Cut | No | CHRC(290)p | PAP1 |
| CHRC | | | observation | as denoted by | as denoted by |
| promoter | | | | SEQ ID NO: 3 | SEQ ID NO: 2 |
| HSP18.2 | PAP1 | Exposure to | No | HSP18.2p | PAP1 |
| promoter | | high | observation | as denoted by | as denoted by |
| | | temperature | | SEQ ID NO: 4 | SEQ ID NO: 2 |
| | | for a | | | |
| | | pre- | | | |
| | | determined | | | |
| | | time (heat | | | |
| | | shock) | | | |
| HSP70B | PAP1 | Exposure to | No | HSP70Bp | PAP1 |
| promoter | | high | observation | as denoted by | as denoted by |
| | | temperature | | SEQ ID NO: 5 | SEQ ID NO: 2 |
| | | for a | | | |
| | | pre- | | | |
| | | determined | | | |
| | | time (heat | | | |
| | | shock) | | | |
| LacIBD: | PAP1 | Exposure to | Observed in | HSP18.2p: | PAP1 |
| Gal4AD | | high | Callus stage | LacIBD: | as denoted by |
| (trans- | | temperature | | Gal4AD:HSP18.2t | SEQ ID NO: 2 |
| cription | | for a | | as denoted by | |
| activating | | pre- | | SEQ ID NO: 6 | |
| sequence) + | | determined | | Lac operator | |
| HSP18.2 | | time (heat | | (X6 LacI | |
| promoter | | shock) | | binding site) | |
| | | | | as denoted by | |
| | | | | SEQ ID NO: 7 | |
| LacIBD: | PAP1 | Exposure to | Observed in | HSP18.2p: | PAP1 |
| NLS:VP64 | | high | Callus stage | LacIBD:NLS: | as denoted by |
| (trans- | | temperature | | VP64:HSP18.2t | SEQ ID NO: 2 |
| cription | | for a | | as denoted by | |
| activating | | pre- | | SEQ ID NO: 8 | |
| sequence) + | | determined | | | |
| HSP18.2 | | time (heat | | | |
| promoter | | shock) | | | |
| LacIBD: | Rosea, Delila | Exposure to | Observed in | HSP18.2p: | Delila |
| Gal4AD | and PAP1 | high | cut leaves | LacIBD: | as denoted by |
| (trans- | | temperature | and matured | Gal4AD:HSP18.2t | SEQ ID NO: 9; |
| cription | | for a | plants in | as denoted by | Rosea |
| activating | | pre- | exposure to | SEQ ID NO: 6 | as denoted by |
| sequence) + | | determined | heat and also | | SEQ ID NO: 10 |
| HSP18.2 | | time (heat | due to a cut | | |
| promoter | | shock) | | | |
| VP64 | Rosea, Delila | Exposure to | Observed in | HSP18.2p: | PAP1 |
| (trans- | and PAP1 | high | cut leaves | LacIBD:NLS: | as denoted by |
| cription | | temperature | and matured | VP64:HSP18.2t | SEQ ID NO: 2; |
| activating | | for a | plants in | as denoted by | Delila |
| sequence) + | | pre- | exposure to | SEQ ID NO: 8 | as denoted by |
| HSP18.2 | | determined | heat and also | | SEQ ID NO: 9; |
| promoter | | time (heat | due to a cut | | Rosea |
| | | shock) | | | as denoted by |
| | | | | | SEQ ID NO: 10 |
| GR:LacIBD: | Rosea, Delila | Exposure to | Significantly | 35Sp:GR:LacIBD: | PAP1 |
| Gal4AD | and PAP1 | dexamethas | observed | Gal4AD:HSP18.2t | as denoted by |
| (trans- | | one | | as denoted by | SEQ ID NO: 2; |
| cription | | hormone | | SEQ ID NO: 11 | Delila |
| activating | | | | | as denoted by |
| sequence) + | | | | | SEQ ID NO: 9; |
| 35S | | | | | Rosea |
| promoter | | | | | as denoted by |
| | | | | | SEQ ID NO: 10 |
| GR:LacIBD: | Rosea, Delila | Exposure to | Significantly | 35Sp:GR:LacIBD: | PAP1 |
| NLS:VP64 | and PAP1 | dexamethas | observed | NLS: | as denoted by |
| (trans- | | one | | VP64:HSP18.2t | SEQ ID NO: 2; |
| cription | | hormone | | as denoted by | Delila |
| activating | | | | SEQ ID NO: 12 | as denoted by |
| sequence)+ | | | | | SEQ ID NO: 9; |
| 35S | | | | | Rosea |
| promoter | | | | | as denoted by |
| | | | | | SEQ ID NO: 10 |

The HSP:PAP1 transgenic plants were exposed daily to a temperature of 50° C. for an hour, during a period of one month. In the leaves of the CHRC:PAP1 transgenic plants, cuts were made in their width and length. During the growth period, no accumulation of anthocyanin was exhibited at any part of the plant and the leaves remained green.

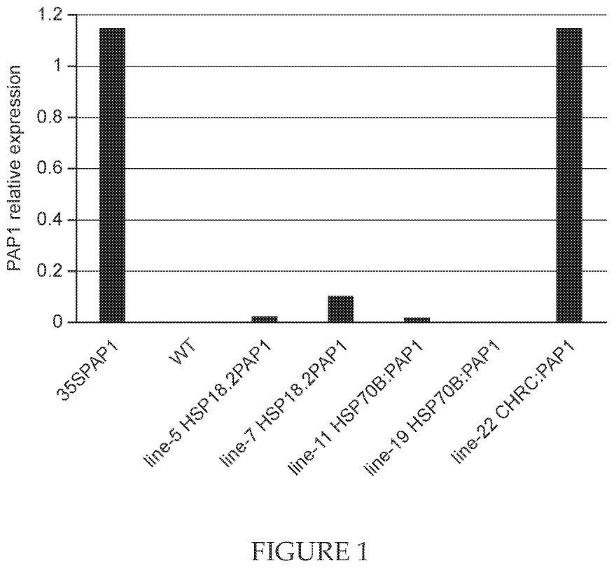

It was then hypothesized that the limiting parameter for production of anthocyanins was the low level of expression of the promoters that were used. In order to examine the level of expression of the gene PAP1 in the HSP:PAP1 and CHRC:PAP1 transgenic plants, a real-time PCR analysis was performed for several lines. For comparison, the level of PAP1 in 35S-PAP1 transgenic plants was employed (FIG. 1). It was shown that the expression level of PAP1 in HSP:PAP1 and CHRC:PAP1 plants was very low in comparison with the control plant (PAP1 activated by 35S) in which the expression level was 10 times higher (FIG. 1).

### Example 2

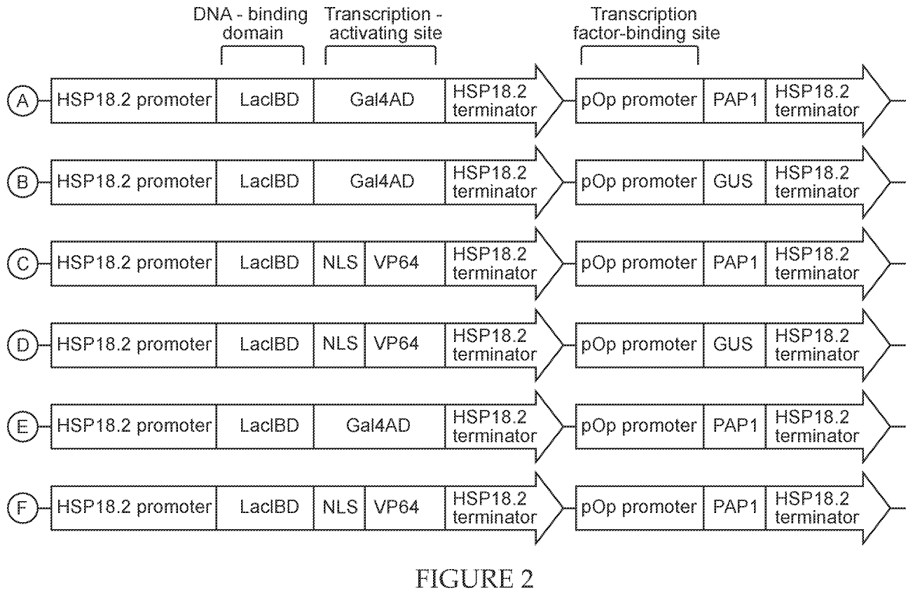

In order to increase the expression level of the PAP1 gene in response to induction, a transactivation system was constructed based on the systems pOp/LhG4 and XVE [4]. In these systems, the target gene is activated by a synthetic transcription factor composed by a DNA-binding domain and a transcription activating site (see FIG. 2). A specific sequence positioned upstream to the target gene (pOp promoter) allows the binding of the synthetic transcription factor. Upon binding of the transcription factor (Lacl) to pOp sequence, the expression of the target gene is enabled by the transcription-activating site. The systems that were constructed, were based on two types of synthetic proteins: in the first type of protein, the transcription activating site of Gal4 is employed and in the second type of protein, the Nuclear Localization Sequence (NLS) was used and fused to the VP64 protein that has four repetitions of the transcription activating site of the VP16 protein [5]. Each of the transcription activating sites was fused to the DNA binding site of the Lacl protein (FIG. 2). In the two systems, the sequence encoding the synthetic protein was constructed under the HSP18.2 promoter or the 35S promoter as a control for the activity of the system in the activation of the anthocyanin pathway. In addition to the PAP1 gene and the GUS reporter gene was used as a control for the activity of the HSP18.2 promoter (FIG. 2).

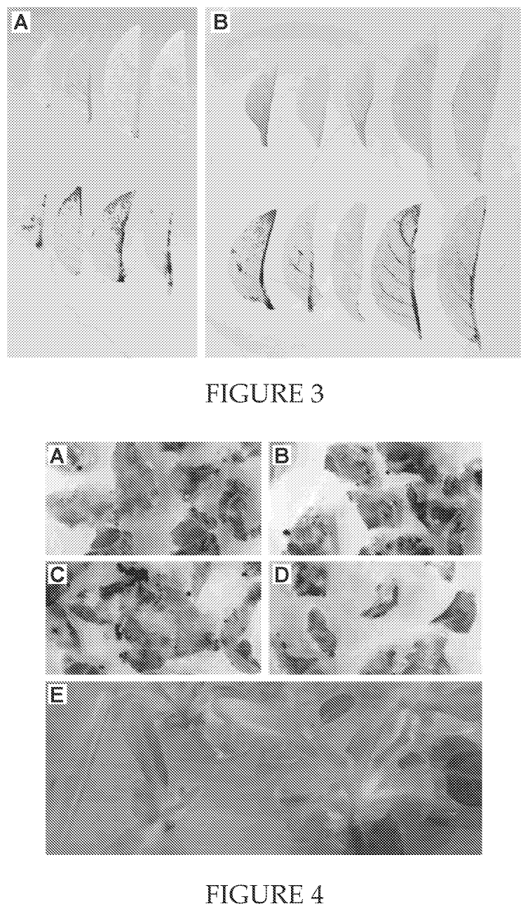

In order to examine the function of the constructed systems, Tobacco transgenic plants were produced including the pOp:GUSgene with different types of transcription factors (FIG. 2). Leaves from the transgenic plants were examined for GUS activity, with or without heat shock. As shown in FIGS. 3A-3B, induction by heat shock led to the activation of GUS gene.

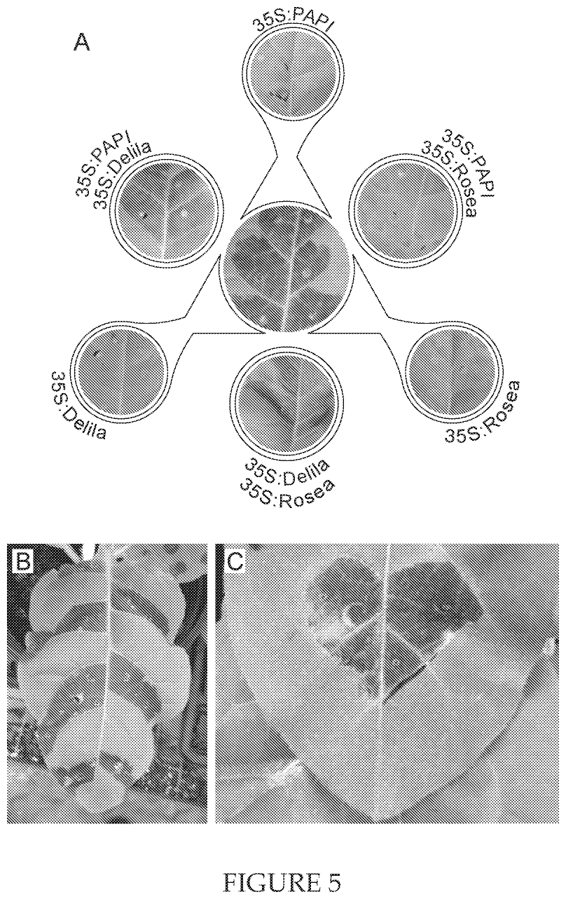

The constructs that included pOp:PAP1 gene were used for transformation into tobacco leaves with agrobacterium. Following 10 days in tissue culture, callus that accumulated anthocyanins in all systems were observed, both wherein the transcription factor was under the 35S promoter and under the HSP18.2 promoter. Accumulation of anthocyanins in the 35S systems was found also in plumules, however in the HSP18.2 systems, the accumulation was limited to the callus stage, namely the plumules that developed afterward were green (see FIGS. 4A-E). Furthermore, the accumulation of anthocyanins was more common in the construct that included the transcription protein with the activating site Gal4. Without being bound by theory, it is possible that the accumulation of anthocyanins that occurred at the callus stage was caused by the fact that in undifferentiated cells, there is no heavy control of the activity of the promoters. Since there were more callus that accumulates anthocyanins when using the Gal4 activation, it seems that the efficiency is higher when using Gal4 in comparison with VP64. In order to examine whether the young plants in the two systems HSP18.2/pOp:PAP1 are able to accumulate anthocyanins following a heat shock, the transgenic plants were exposed to a temperature of 37° C. for an hour, two hours or 16 hours. No anthocyanins accumulation was observed.

### Example 3

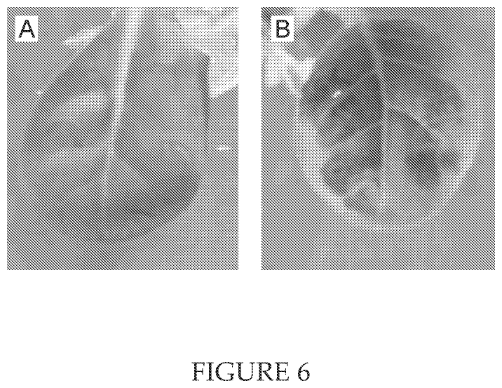

To investigate whether the accumulation of anthocyanins in the tobacco tissues requires simultaneous expression of several genes known to belong to the complex that includes also PAP1, binary plasmids that include the genes 35S:PAP1, 35S:Rosea and 35D:Delila were constructed and introduced into agrobacteria. Infiltration of these three types of agrobacteria into the leaves was carried out in different combinations or separately. Following 4 days, accumulation of anthocyanins was observed in the leaves infected with the agrobacteria that carried the genes 35S:PAP1 or 35S:Rosea in combination with 35S: Delila. No anthocyanins accumulation was observed in leaves infected with the combination of 35S:PAP1 with 35S:Rosea or each of them alone (see FIG. 5A). Significant accumulation of anthocyanins was observed in the leaves that were infected with the combination of the three genes, thereby allowing to obtain clear patterns on the leaves (see FIG. 5B-5C).

### Example 4

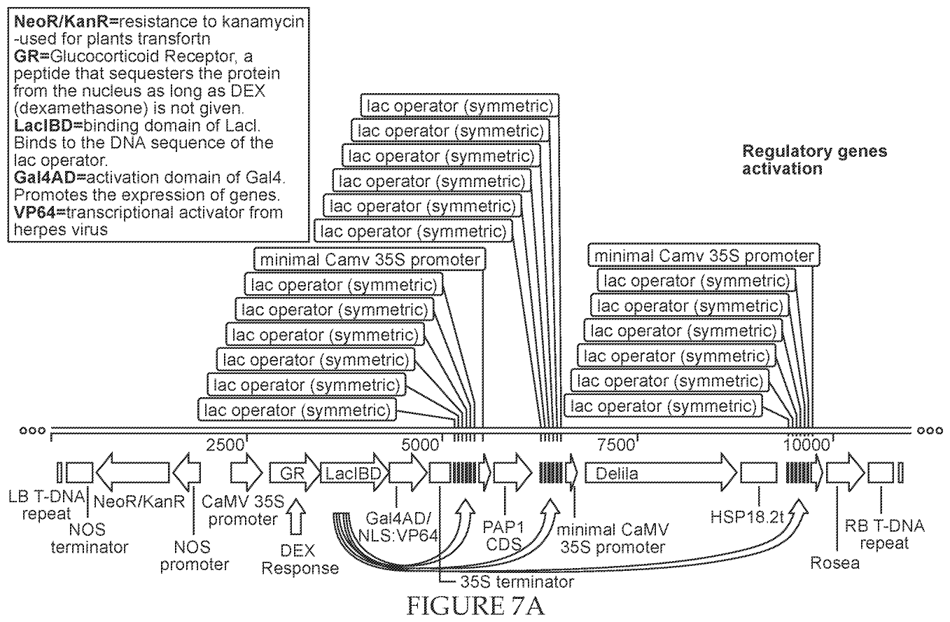

In order to examine the accumulation of pigments as a result of induction by heat shock in tobacco plants, infiltration of agrobacteria carrying the two genes 35S:Rosea/35S: Delila into leaves of HSP18.2/pOp:PAP1 transgenic lines was performed. The infiltration was performed in transgenic plants that were treated with a heat shock overnight and in transgenic plants that were not. After about four days, significant accumulation of anthocyanins was observed in the leaves of lines that were exposed to a heat shock (see FIGS. 6A-B). In order to promote expression of the three genes PAP1, Rosea and Delila following induction by a heat shock, systems were constructed including these genes and the promoters detailed above. These systems were used to transform Tobacco leaves. Activity assay of these systems is performed in adult transgenic plants.

### Example 5

A set of plasmids was established carrying genes that are responsible for anthocyanin production (Rosea/Delila/PAP1/F3H (the latter was taken from Petunia X hybrida). These genes are transactivated by a synthetic transcription activator that can be induced by a chemical (i.e. Dexamethason) (FIG. 7A) or by heat shock (FIG. 7B). The anthocyanin biosynthetic gene F3H was also placed downstream to a heat shock sensitive promoter and can be directly induced (FIG. 7C).

### Example 6

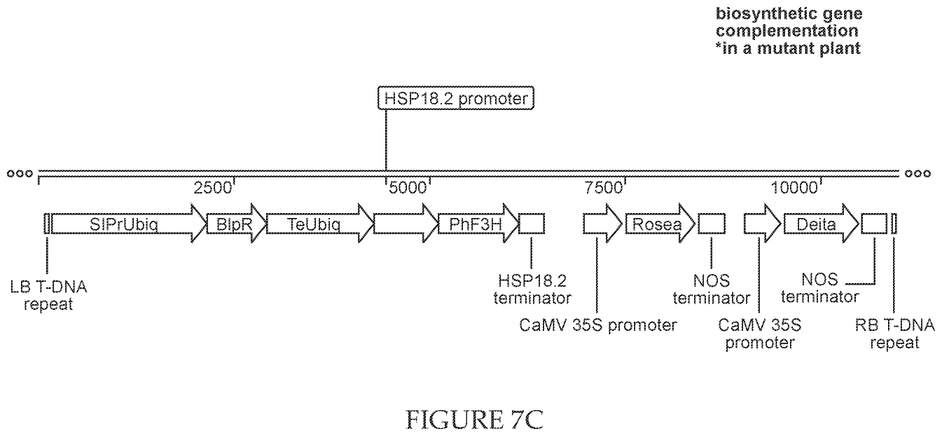

The above described plasmid systems have been introduced also into tobacco plants with mutated anthocyanin pathway gene, flavanone 3β-hydroxylase (F3H), in order to minimize background anthocyanin production resulting from the induction by transcription factors described above. To initiate pigmentation with the designed pattern, the active version of the mutated anthocyanin pathway gene F3H under inducible promoter was incorporated into these plants. To demonstrate the feasibility of the mutant system, 35S:Rosea/35S: Delila and 35S:F3H were transiently expressed in leaves which led to anthocyanin accumulation. Anthocyanin did not accumulate in leaves expressing 35S:Rosea/35S: Delila (see FIG. 8).

### Example 7

### Experiment Description—

Tobacco plants capable of significant anthocyanin accumulation upon induction were produced as described above in Example 1. The Tobacco plants were having the construct as described in FIG. 7A. The plants were grown for about a month in a greenhouse and gained impressive biomass. Then the growth media was dried for two days and on the third day, each plant was irrigated with 1 Liter solution containing water and dexamethasone at a 10 μM concentration. After the induction, leaves from each plant were sampled every day. Control samples were collected from the following plants: wild-type tobacco plant, a modified plant according to the invention without induction and transgenic plants expressing the same genes (Rosea/Delila/PAP1) under overexpression conditions under the control of 35S. The various samples of leaves were dried using liquid nitrogen and crushed to a paste with a mortar & pestle. Subsequently, the samples underwent a double extraction with acidic ethanol at 40° C., the solid parts were separated by centrifugation. The samples were then tested using a spectrophotometer.

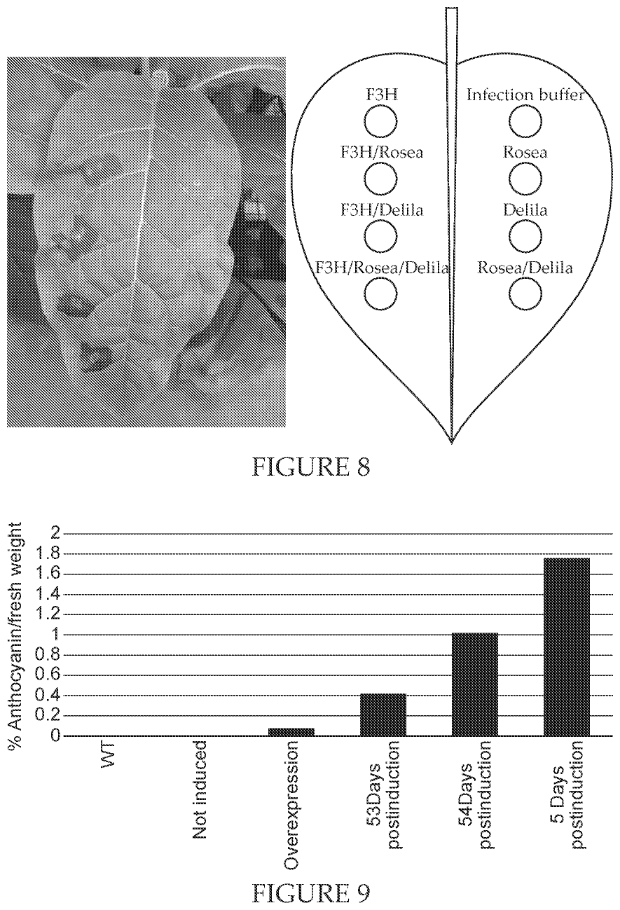

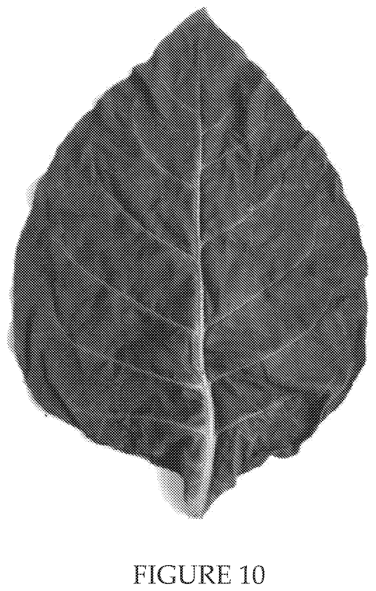

The anthocyanin concentration in the leaves of the modified plants of the invention were rising continuously until the fifth day post induction (FIG. 9). On the fifth day the concentration was 1.75% of the fresh weight, which is considered a very high result (FIG. 10). These results demonstrate that the method of the invention is significantly more effective in achieving high expression of anthocyanin than that achieved by plants expressing the same genes under overexpression conditions.

### Example 8

The following example concerns the induced biosynthesis of valencene. Valencene is a sesquiterpene that is an aroma component of citrus fruit and citrus-derived odorants.

The following enzymes are required for valencene biosynthesis:

The enzyme 3-hydroxy-3-methylglutaryl-coenzyme A reductase (HMG-R), for example the yeast Hmg1p, catalyzes biosynthesis of mevalonate. To enhance mevalonate production, a mutated form can be used. This form of HMG-R (tHMG) has its N-terminal-truncated. This truncation results in a soluble form of the enzyme that is not subjected to inhibition by the pathway's products and hence causes high production levels.

The enzyme farnesyl pyrophosphate synthetase (FPPS), for example the Arabidopsis AtFPPS, catalyzes biosynthesis of FPP from the compound IPP. The latter derives from mevalonate.

The enzyme Valencene synthase (TPS), for example CsTPS1 (derived from Citrus sinensis)—catalyzes biosynthesis of Valencene from the compound farnesyl pyrophosphate (FPP).

Valencene biosynthesis can occur within the cell's cytosol, mitochondria, or chloroplasts. Thus, a corresponding peptide signal can be fused to the genes to target different organelles.

A detailed description of the plasmids encoding for valencene induced biosynthesis is provided in Table 2.

| | | | | |
|---|---|---|---|---|
| LacIBD:Gal4AD | AtFPPS, | Exposure to | HSP18.2p:LacIBD: | AtFPPS as |
| (transcription | tHMG and | high | Gal4AD:HSP18.2t | denoted by |
| activating | CsTPS1 | temperature | as denoted by | SEQ ID NO: 13; |
| sequence) + | | for a | SEQ ID NO: 6 | tHMG as |
| HSP18.2 | | predetermined | Lac operator | denoted by |
| promoter | | time (heat | (X6 LacI | SEQ ID NO: 14; |
| | | shock) | binding site) | CsTPS1 as |
| | | | as denoted by | denoted by |
| | | | SEQ ID NO: 7 | SEQ ID NO: 15 |
| LacIBD:NLS: | AtFPPS, | Exposure to | HSP18.2p:LacIBD: | AtFPPS as |
| VP64 | tHMG and | high | NLS: | denoted by |
| (transcription | CsTPS1 | temperature | VP64:HSP18.2t | SEQ ID NO: 13; |
| activating | | for a | as denoted by | tHMG as |
| sequence) + | | predetermined | SEQ ID NO: 8 | denoted by |
| HSP18.2 | | time (heat | | SEQ ID NO: 14; |
| promoter | | shock) | | CsTPS1 as |
| | | | | denoted by |
| | | | | SEQ ID NO: 15 |
| VP64 | AtFPPS, | Exposure to | HSP18.2p:LacIBD: | AtFPPS as |
| (transcription | tHMG and | high | NLS: | denoted by |
| activating | CsTPS1 | temperature | VP64:HSP18.2t | SEQ ID NO: 13; |
| sequence) + | | for a | as denoted by | tHMG as |
| HSP18.2 | | predetermined | SEQ ID NO: 8 | denoted by |
| promoter | | time (heat | | SEQ ID NO: 14; |
| | | shock) | | CsTPS1 as |
| | | | | denoted by |
| | | | | SEQ ID NO: 15 |
| GR:LacIBD: | AtFPPS, | Exposure to | 35Sp:GR:LacIBD: | AtFPPS as |
| Gal4AD | tHMG and | dexamethasone | Gal4AD:HSP18.2t | denoted by |
| (transcription | CsTPS1 | hormone | as denoted by | SEQ ID NO: 13; |
| activating | | | SEQ ID NO: 11 | tHMG as |
| sequence) + | | | | denoted by |
| 35S promoter | | | | SEQ ID NO: 14; |
| | | | | CsTPS1 as |
| | | | | denoted by |
| | | | | SEQ ID NO: 15 |
| GR:LacIBD: | AtFPPS, | Exposure to | 35Sp:GR:LacIBD: | AtFPPS as |
| NLS:VP64 | tHMG and | dexamethasone | NLS:VP64: | denoted by |
| (transcription | CsTPS1 | hormone | HSP18.2t | SEQ ID NO: 13; |
| activating | | | as denoted by | tHMG as |
| sequence) + | | | SEQ ID NO: 12 | denoted by |
| 35S promoter | | | | SEQ ID NO: 14; |
| | | | | CsTPS1 as |
| | | | | denoted by |
| | | | | SEQ ID NO: 15 |

1-63. (canceled)

64. A genetically modified plant, comprising a recombinant nucleic acid molecule that encodes for at least one gene, wherein upon controllable induction of expression of said gene in one or more parts thereof, said genetically modified plant exhibits a phenotypic change at a predesigned location;
wherein said recombinant nucleic acid molecule further comprises a binding site for a transcription factor of said gene; and
wherein the phenotypic change is an expression of any one of: enzymes involved in pigments, metabolites and terpenes.

65. The genetically modified plant of claim 64, wherein the phenotypic change is a color change, luminosity, or production of a compound.

66. The genetically modified plant of claim 64, wherein said at least one gene is a regulatory gene, a gene encoding a protein involved in plant pigmentation, a chromoprotein encoding gene, a gene encoding a protein involved in bioluminescence, a gene encoding an enzyme involved in the production of a compound selected from a group consisting of: flavonoids (e.g. anthocyanidins, flavanols, flavones, flavonols, flavonones or isoflavones), betalains, chromoproteins, Terpenes (e.g. monoterpenes (genipin, limonene) (C₁₀), sesquiterpenes (valencene, artimesnin) (C₁₅), diterpenes (retinol, retinal, Taxol, and phytol) (C₂₀), Sesterterpenes (Rebaudioside A, Merochlorin A, Ophiobolin A, Ophiobolin B, Ophiobolin C) (C₂₅), Triterpenes (Mogrosides, Squalene, α-Amyrin, β-Amyrin, Cucurbitacin B, Sitosterol, Stigmasterol, Campesterol, α-Spinasterol) (C₃₀), Carotenoids (Phytofluene, Lycopene, Cynthiaxanthin, Pectenoxanthin, Lutein, Zeaxanthin, Glycosides, Astaxanthin) (C₄₀), Alkaloids (e.g. Ajmalicine, Ajmaline, Berberine, Camptothecin, Capsaicin, Capsorubin, Codeine, Colchicine, Ellipticine, Emetine, Morphine, Quinine, Sanguinarine, Vincristine, Vinblastine), or a gene encoding a protein (e.g. albumin, globulin, glutelin, Melatonin).

67. The genetically modified plant of claim 64, wherein the induction of expression of said at least one gene is performed by local activation and wherein said activation is selected from high temperature, exposure to light characterized by a certain wavelength, injuring, application of response inducing material or a combination thereof.

68. The genetically modified plant of claim 67, wherein said response inducing material is selected from the group consisting of Gibberellin hormone, dexamethasone, abscisic acid (ABA), β-Aminobutyric Acid (BABA), ethanol, auxin, cytokinin (CK), strigolactone, salicylic acid, protocatechuic acid (PCA), Vanilic acid (VA), phloretin and any combination thereof.

69. The genetically modified plant of claim 64, wherein the phenotypic change is due to expression in the plant part of any one of the group consisting of: flavonoids (e.g. anthocyanidins, flavanols, flavones, flavonols, flavonones or isoflavones), betalains, chromoproteins, Terpenes (e.g. monoterpenes (genipin, limonene) (C₁₀), sesquiterpenes (valencene, artimesnin) (C₁₅), diterpenes (retinol, retinal, Taxol, and phytol) (C₂₀), Sesterterpenes (Rebaudioside A, Merochlorin A, Ophiobolin A, Ophiobolin B, Ophiobolin C) (C₂₅), Triterpenes (Mogrosides, Squalene, α-Amyrin, β-Amyrin, Cucurbitacin B, Sitosterol, Stigmasterol, Campesterol, α-Spinasterol) (C₃₀), Carotenoids (Phytofluene, Lycopene, Cynthiaxanthin, Pectenoxanthin, Lutein, Zeaxanthin, Glycosides, Astaxanthin) (C₄₀), Alkaloids (e.g. Ajmalicine, Ajmaline, Berberine, Camptothecin, Capsaicin, Capsorubin, Codeine, Colchicine, Ellipticine, Emetine, Morphine, Quinine, Sanguinarine, Vincristine, Vinblastine), proteins (e.g. albumin, globulin, glutelin, Melatonin) and any combination thereof.

70. The genetically modified plant of claim 64, wherein said at least one gene is selected from a group consisting of the LC gene from maize, the C1 gene from maize, the PAP1 gene from Arabidopsis, the Rosea gene from Antirrhinum, the Delila gene from Antirrhinum, flavanone 3β-hydroxylase (F3H), HMG-R gene from yeast, FPPS gene from Arabidopsis, TPS gene from Citrus or any combination thereof.

71. The genetically modified plant of claim 64, wherein said gene is a gene encoding a protein involved in plant pigmentation, and wherein at least one endogenous gene responsible for pigmentation in said plant is knocked-out.

72. The genetically modified plant of claim 64, wherein the induction of expression of said gene is performed when the plant is fully grown.

73. A method for generating a genetically modified plant comprising:
(a) transforming a plant cell in a plant tissue or plant part with a recombinant nucleic acid molecule encoding at least one gene, wherein said at least one gene is capable of affecting a phenotypic change in a plant part; and wherein upon induction of expression of said gene, said genetically modified plant exhibits a visible phenotypic change at the location of the induction, wherein said recombinant nucleic acid molecule further comprises a binding site for a transcription factor of said gene, and wherein the phenotypic change is an expression of any one of: enzymes involved in pigments, metabolites and terpenes;
(b) regenerating a transformed plant from said plant cell; and
(c) performing induction of said at least one gene when the plant is fully grown.

74. The method of claim 73, wherein said gene is a regulatory gene, a gene encoding a protein involved in plant pigmentation, a chromoprotein encoding gene, or a gene encoding a protein involved in bioluminescence.

75. The method of claim 74, wherein said at least one gene is a regulatory gene involved in anthocyanin gene expression.

76. The method of claim 75, wherein said at least one gene is selected from a group consisting of the LC gene or the C1 gene from maize, the PAP1 gene from Arabidopsis, and the Rosea gene or the Delila gene from Antirrhinum, flavanone 30-hydroxylase (F3H), petunia AN1, AN2, AN11, Tomato Myb12, Vitis vinifera and any combination thereof.

77. The method of claim 73, wherein said transforming step is performed by transfecting an agrobacterium with at least one inducible plasmid, wherein said at least one inducible plasmid comprises (i) a promoter operatively linked to a nucleic acid sequence encoding a synthetic transcription protein comprising a DNA binding domain and a transcription activating domain, and (ii) multiple operator sequences operatively linked to at least one promoter said promoter being further operatively linked to at least one gene.

78. The method of claim 77, wherein said plasmid further comprises a sequence encoding NeoR/KanR and/or a sequence encoding glucocorticoid receptor (GR).

79. The method of claim 77, wherein said at least one promoter is selected from the group consisting of the carotenoid associated gene (CHRC) promoter, a truncated CHRC promoter, heat shock protein (HSP) 18.2 promoter, HSP 70B promoter, the cauliflower mosaic virus (CaMV) 35S promoter and minimal CaMV 35S promoter.

80. The method of claim 77, wherein:
said DNA binding domain is LacIBD;
the operator is lac operator; and/or
said transcription activating domain is Gal4AD or VP64 optionally linked to a nucleus localization sequence (NLS).

81. The method of claim 73, wherein at least one endogenous gene responsible for pigmentation in said plant is knocked-out and wherein said recombinant nucleic acid comprises at least one gene encoding a protein involved in plant pigmentation.

82. The method of claim 81, wherein said at least one gene encoding a protein involved in plant pigmentation is selected from the group consisting of anthocyanins, flavonoids, carotenoids, betalains, chromoproteins, and any combination thereof.
